(19) United States
(12) Patent Application Publication
(10) Pub. No.: US 2021/0353801 A1
(43) Pub. Date: Nov. 18, 2021

(54) Autonomous Decontamination Device for the Dispersion of Disinfecting, Sanitizing and Barrier Agents for Enclosed Structures

(71) Applicant: Kent Weisenberg, Fruit Cove, FL (US)

(72) Inventor: Kent Weisenberg, Fruit Cove, FL (US)

(21) Appl. No.: 17/223,560

(22) Filed: Apr. 6, 2021

Related U.S. Application Data
(60) Provisional application No. 63/005,685, filed on Apr. 6, 2020.

(51) Int. Cl. A61L 2/20; A61L 2/24

(52) U.S. Cl. CPC A61L 2/20; A61L 2/24; A61L 2202/14; A61L 2202/15; A61L 2202/11

(57) ABSTRACT

An autonomous device for the vaporization and dispersal of a sanitizing agent adapted for the use in closed structures such as rooms, vehicles, trash bins, dumpsters, and other containers.

### BACKGROUND OF THE INVENTION

The invention relates generally to the field of devices for delivering disinfecting, sanitizing and/or barrier agents (to be referred to herein collectively as “sanitizing agents”) into the interior of closed structures, containers, receptacles or similar objects, and more particularly relates to such devices that are autonomous and which disperse the agents in vaporized form into the closed structures. As used herein, the term “vaporization” or related grammatical versions of the term shall be taken to mean the agent undergoes a phase transition, e.g., a change from the liquid phase to the vapor phase through evaporation, a surface phenomenon, or boiling, a bulk phenomenon.

There are many known compositions and the applications thereof for decontaminating containers and surfaces. There are however numerous problems or drawbacks associated with the known compositions and methodologies.

Furthermore, all the devices that are used currently for the dispersion of these sanitizing agents are manually controlled by humans which involves manual spraying, atomization or wiping of the sanitizing agent to provide coverage on surfaces (basically human aiming, wiping or spraying of the agent through a variety of means). This significantly increases the potential for applicator/human error in achieving full coverage on surfaces.

The method utilized by current devices is dispersion of a single trajectory stream or spray pattern (spray bottle, garden pump up sprayer, electric automizer, spray gun etc.) which does not insure full coverage, i.e., encapsulation, of the agent to all surfaces in the enclosed structure. Streaming/spraying/atomization/aerosolization/wiping will not get in and around profiles on surfaces such as garbage bags, boxes, beds, chairs, faucets etc. In spraying, aerosolizing or atomization techniques the sanitizing agent is dispersed in small droplets in conjunction with air, which decreases the surface coverage effectiveness due to the spaces between the droplets on a surface.

Also, the known application practices of these sanitizing agents are typically not effective in the eradication and or repelling of all relevant pathogens, viruses, bacteria, mold, fungi, insects, larvae and animals. Current compositions of these sanitizing agents are typically not effective in the eradication and or repelling of all relevant pathogens, viruses, bacteria, mold, fungi, insect, larvae or animals. Most compositions of these sanitizing agents do not create a sustaining barrier on surfaces to arrest all potential for new pathogen life and growth and do not create a sustaining repellent to insects and animals (i.e., they do not create barrier for up to 24 hours from reinfection). Moreover, the compositions of these sanitizing agents are not approved for food contact by the FDA and are hazardous to both humans and the environment (whereas the components used herein are most preferably botanical or other compounds occurring in nature). Neither do they create a pleasing sustaining aroma (which is accomplished herein using pure, sustainable, essential oils such as peppermint, lavender etc.).

This invention, while applicable in multiple situations to address viruses, bacteria, fungi mold and other pathogens or undesirable biologics, is well adapted to address the coronavirus pandemic. Beginning in December 2019, the 2019-20 coronavirus pandemic is an ongoing pandemic of coronavirus disease 2019 (COVID-19), caused by severe acute respiratory syndrome coronavirus 2 (SARS-CoV-2). As of 2 Apr. 2020, more than 965,000 cases of COVID-19 had been reported in over 200 countries and territories, resulting in over 49,200 deaths. Similar events happened in history like Spanish flu in 1918, SARS in 2003, MERS in 2012, Ebola virus in 2013, and the Seasonal H3N2 flu, all these extremely contagious diseases caused by virus have taken millions of people's lives and a tremendous losses in the economy all over the world. Many of these contagious viruses are spread during close contact and by small droplets produced when people cough or sneeze. Respiratory droplets may be produced during breathing, but the viruses are not generally airborne. It is now believed by the scientific community that the main cause of spread is through the touching of a contaminated surface and then transferred by touching the eyes, mouth or nose. The virus is most contagious when people are symptomatic, although spread has been noted before symptoms appear. Recommended preventive measures include hand washing, covering one's mouth when coughing, maintaining safe distance from other people, and monitoring and self-isolation for people who suspect they are infected.

Many of the countries ask their citizens to isolate themselves from public or do self-quarantine for weeks or even months to slow down the virus spread. Specific in the daily maintenance of people's lives and livelihoods, they need to get their mail, order daily necessities and food online which is then delivered to their homes or businesses. However, since the virus is so contagious and widespread, people have no way to verify that these envelopes, packages, goods and food they receive has remained safe and virus free.

The coronavirus responsible for the disease COVID-19 can remain intact on surfaces for anywhere up to 72 hours, according to a study conducted by US researchers. When on plastic, coronavirus strains appear to be able to stay intact far longer. Only half of the SARS-CoV-2 particles broke apart in just under seven hours, for example, with viable coronavirus particles were still detected up to three days later. Stainless steel was almost as bad, with a half-life for SARS-CoV-2 of 5.6 hours. Copper, on the other hand, failed to provide similar protection for either strain, with the number of viable particles capable of causing disease vanishing within just four hours for SARS-CoV-2, and eight hours for SARS-CoV-1. Similarly, on cardboard, no viable SARS-CoV-2 particles could be found after 24 hours, or SARS-CoV-1 after eight. There are still several variables to keep in mind. Variations in individual results show how much the timing is affected by subtle differences. With millions of packages, mail and other items being shipped and delivered through US each day it is imperative to sanitize these items prior to human contact in a concerted attempt to arrest the spread of the virus. Because of the long active period of Coronavirus on cardboard, to avoid bringing the virus into their homes people may need to leave their packages outside for days before the virus breaks down and is eradicated. This is unsafe and impractical for food and even everyday life.

Some people do attempt to sanitize their packages as precautions with wipes and sprays; however, the general available sanitizing methods and agents are not very effective way for treating these surfaces in general. Many of the current sanitizing agents are not effective in the eradication and or repelling of all relevant pathogens, viruses, bacteria, etc., nor are approved for food contact or human direct contact. Additionally, most of the sanitizing devices, such as a sprayer bottle, sanitizing wipes, and even UV lights, are significantly dependent on the efficacy of the applicators, significantly increasing potential human error in achieving full coverage on package surfaces.

A new safe and effective sanitizing method is urgently needed now for people and businesses to fully sanitize their delivered mail and packages quickly and thoroughly. The innovative sanitizing method disclosed herein of using vaporized sanitizing agents in an enclosed structure or container addresses these problems.

### SUMMARY OF THE INVENTION

An autonomous non-toxic sanitizing agent vaporization and dispersal device has been developed for the use in trash bins, dumpsters, containers, rooms or other such enclosed structures. This invention relates generally to the sanitizing and or odor and pest control and or repelling insects, animals and humans and more particularly relates to sanitizing and or odor and pest control of surfaces, containers, and even more particularly relates to trash bins, dumpsters, shipping containers, portable decontamination boxes and other such enclosed structures. Such invention can be adapted and adaptable for use for any enclosed structure for the decontamination of surfaces affected or potentially affected with any virus, bacteria, fungi, mold or insect larvae, whether the surfaces are part of the enclosed structure itself or are surfaces on objects, items, containers, packages, trash, etc. placed into the enclosed structures. The invention further relates to methods for applying a long-lasting repellent and barrier to sustainable life or growth these substances.

Multiple modes of control are provided including a handheld rf-remote control, timer/clock-based scheduling, over Bluetooth with a mobile device, over an internet connected device with a web browser, over the internet with a custom iOS and Android application. The sanitizing agents are 99.9% effective in the eradication of viruses, bacteria, fungi, mold or insect larvae on any surface. By vaporizing the sanitizing agent there is complete engulfment of objects within an enclosed structure.

This new sanitizing method involves an autonomous vaporizing system which can vaporize and dispense sanitizing agents, such as thyme oil (thymol), isopropyl alcohol, botanicals or other types of agents suspended in glycerin to fully engulf the goods or packages on all surfaces. The system can vaporize the agents in a few seconds and dispense and circulate the agents through all the exposed surface to assure contact in less than a minute or a few minutes depending on the size of the structure or container.

The entire sanitizing process will be active for about 10 minutes more or less depending on the container or structure size and agent being used. Considering all preferred agents used in this method are FDA approved for food contact there are no harmful residuals on the sanitized surfaces to be removed prior to handling.

The vaporizing systems can either have one or multiple sanitizing agent containers to stock various agents for different application purposes, including sanitizing, cleaning odors, control molds, etc. Each container can retain enough agent for a typical serving period of three to six months depending on the application frequency. This system can be installed in containers such as coolers, lined cardboard boxes or similar that will be put in delivery places such as in a mailbox or mailbox area, front porch or shipping dock. The delivery staff will leave the packages in the containers and close the container lids. Customers or delivery personnel then can activate the vaporizing system via a small keychain remote control on to fully sanitize the packages without ever touching them. The entire sanitizing process can also be done through smartphone apps. The sanitizing system utilizes batteries as the power source which can last up to least six months in general depending on the application frequency. This allows the portable sanitizing systems to be installed almost everywhere even those places without direct electric power.

For applications in enclosed structures, such as medical offices, patient rooms, and other places, the system can be permanently installed or mounted in the interior and modified using wall power, as opposed to batteries, and higher power equipment to expedite the vaporizing and sanitizing processes. For ease of discussion, but in no way meant to be limiting, the invention will be discussed in more detail with regard to a device to be installed in small, enclosed portable or transportable structures, which may be portable, such as coolers, cardboard boxes, 5-gallon buckets, trach receptacles, shipping containers, etc. or any such container with a lid. The unit can also be placed in containers that are fixed and normally used for the receipt of delivered items, such as mailboxes, waterproof shipping boxes, porch storage boxes, etc. To promote the full coverage of the agent it is recommended that these containers be fitted with some type of readily available drying grid, fencing or small diameter rods on the bottom of the container and in-between packages if they are to be stacked or layered in the container. This will allow for full contact of vapor with all surfaces. In absence of these commercially available items the container can simply be turned 90 degrees during the fogging process to facilitate the exposure of all surfaces.

In Cities and towns across north America, trash Bins—Wheelie Bins or sometimes called ‘Herbie Curbies’ in the north—are used for garbage collection, recycling and yard waste. In most cases, due to city ordinances, community or HOA guidelines, trash bins are required to be stored out of sight—in garages or specific designated areas right next to homes.

Based on the most current research there are approximately 95 million outside trash bins used by single family homes in the US alone. Of this number nearly 50 million are located and used in residential areas where a homeowner association or deed restrictions afford directives for the storage of these trash bins. Based on the same research there are approximately 125 million US residents living in multi-family residences such as condos and apartments. Typically, these residents dump their interior trash into large dumpsters located onsite. Both single family and multi-family residences need an autonomous method for sanitizing their bins. While trash bins and dumpsters were the vision of this invention this technology can be utilized in many other applications as it is extremely versatile.

One example of this versatility would be utilization in shipping containers of which there are approximately 600 million units globally. This device could be used autonomously to control odor, bacteria, insect infestation in the products shipped worldwide in these containers.

While most people never considered surface contact transfer, recent epidemics and pandemics have sincerely highlighted to the global population of the high probability for transmission of viruses and bacteria from touching of surface contamination through everyday activities. There is a high probability that people will now fully consider surface transmission and govern themselves accordingly.

The underlying threat to residents is that these trash bins accumulate bacteria, viruses, waste residues and odors that are unpleasant as well as being proven harmful to humans. Typically, the homeowner or resident is not interested in taking the time or money to sanitize their bins. When bins are stored in garages and even outdoors, in addition to the noticeable odors, these residents are at risk of exposing their home and families to unsafe bacterial including but not limited to Covid, Listeria, Salmonella and E. coli when they are in contact with these containers.

Though seldom allowed by deed restrictions or HOA rules, some residents store unclean bins near or around the side of their residences. Beyond still being at risk of exposure to odor, bacteria, viruses, the home is exposed to an increased incidence of rodent infestation such as mice, rats and raccoons. These unwelcome creatures find garbage as an excellent source of food.

When residents encounter a trash bin or dumpster and then touch their children it presents the potential for illness or even worse. Salmonella has been known to be one of the dangerous bacteria lingering in, and on, trash bins, as the CDC reports 1.2 million cases of Salmonellosis and 450 deaths per year in the United States alone, many from contact with contaminated surfaces. This highlights a tremendous health benefit to having an adequately cleaned and disinfected trash bin.

Whether plastic bags are used or not, bacteria can be widely spread throughout the trash bin. Plastic materials such as the materials the most popular trash bins are made out of, are proven hold bacteria and viruses much longer than other similar surfaces. Bacteria thrive in this garbage filled cans, especially as temperatures heat up and outside trash can or dumpster is easily one of the most neglected household items used. Every day, uneaten breakfast, lunches and dinner get tossed into the trash to be forgotten—out of sight—out of mind. However, while people surely are accustomed to throwing out the trash bag from their inside can once it becomes smelly, they rarely concern themselves with their outdoor containers, but it is a key to their overall health. People put everything in their trash bins and dumpsters from food waste to pet fecal contaminated matter. While plastic trash bins that are rolled to the curbside each week have made trash storage and disposal easier, they have become the most unsanitary item in the home and workplace. Even after they are emptied, harmful germs, bacteria and food related molds are left behind. Again, in addition to an offensive odor and bacteria, dirty trash cans attract unwanted pests and are a breeding ground for insects such as spiders, ants, and flies.

Most people do not even try to clean their trash can on their own. When they do most often, they simply pour bleach into the bin and fill it part way up with water, scrub with brush, let it sit and then pour it out into the street or driveway. While this will kill active bacteria and viruses the trash bin is only sanitized until the next time garbage is put into it. It should also be noted that it is against federal regulations and violation of law and most city ordinances to allow this cleaning solution to enter the storm drains as it ends up in our lakes and rivers. Cleaning the trash bins can present serious problems. Besides being a human health risk directly related to the harmful bleach residue in the can, there is a negative environmental impact to cleaning these trash cans that most people simply do not consider.

Recently, there have been mobile services start-ups that provide high pressure washing for these trash bins on a weekly, bi-weekly, or monthly basis. They are expensive and relatively ineffective for the reduction in transmittable contaminants. While the cleaning agent and water are contained in this process the water still needs to be disposed on somewhere. More importantly as with self-cleaning, the trash bin or dumpster is only sanitized until the next bag of garbage is put in it. There are many other ‘stick on’ odor remedies and sprays on the market for trash bins. These too are with only effective for masking odors and or only provide a short-term topical solution, “topical” meaning that when sprayed in a trash bin the dispersed agent only lands on surfaces in facing applicator. Covered surfaces or those surfaces underneath are not coated with the spray therefor are ineffective.

After just a couple days of garbage being left out, the harmful bacterium begins to grow. Animals and insects get into the garbage can and spread this disease and bacteria. Most all people notice that something is going on due to how their trash bins smells, but most fail to know what produces those nasty odors. They are produced by strong bacteria growth. Studies show that frequent cleaning (every 2-3 days) is required to reduce the risk of biological infection from waste containers. Effective self-cleaning of trash bins is a hard task to accomplish and one never knows if they have eradicated all the contamination present.

Many strains of bacteria can thrive in trash bins but the most common ones are Salmonella, E. Coli, and Listeria. Salmonella is bacteria usually found in eggs, uncooked poultry or raw meat. Salmonella poisoning can cause diarrhea, nausea, vomiting, fever, headaches and in children, the elderly and people with underlying conditions even death. The common cold and flu viruses can make it into the garbage bin by discarded tissues and napkins that contain the virus. E. Coli is found in uncooked beef and other household foods like cucumber. E. coli infection symptoms include abdominal cramps, diarrhea, tiredness, vomiting and in children, the elderly and people with underlying conditions even death. Listeria can be found in many foods and thrive in colder temperatures.

Maggots are one the most common insects found at the bottom of outside trash bins. They are the larva of various species of flies including houseflies, horseflies and fruit flies. These small white insects feed on decaying food that was inside the bags of trash. Maggots are harmful to harmful not only to human health but that pets as well. One example of the danger in self-cleaning of trash bins is that the maggots getting poured into the yard or driveway and pets ingest them causing illness and sometimes death.

There is no doubt that bacteria live on & inside and outside trash cans. People constantly are throwing away scrap food and every week the trash is picked up by a waste management company headed to the landfill. Regardless, if your trash is protected by a bag, you still run the risk of spreading bacteria. According to studies, a survey shows that there are over 411 million bacteria per square inch on an average family home's bin. To compare, that's more bacteria than what lives on your bathroom light switch, toilet seat and flush handle.

Viruses are harder to eradicate than bacteria born illness. Certain viruses, including those that cause the cold or flu, can survive for 48 hours outside of the human body. Trash bins can host them and since viruses live longer on non-porous surfaces, like trash bins, without cleaning it regularly, one exponentially increases the chances for becoming infected.

Not only does this invention continually and autonomously keep the homeowner and business owner safe from these infectious diseases it protects the service provider personnel with added safety and well being as well. These sanitation workers are exposed to these contaminants daily through contact with the trash bins as well as being in contact with the storage of the garbage. This device when used would eliminate the potential for exposure to both the people that handle the consumers garbage but also their families.

One other item of note is that the use of this invention and the process and agents therein would impact and reduce the catalysts of climate change. Due to the decomposing meat, bacteria, feces etc. in trash bins and dumpsters a small amount of methane gas and carbon dioxide emit from each trash bin every day in the US and the world. The inherent result in the utilization of the device and its agents arrests this decomposition and bacterial growth process thereby significantly reducing or eliminating the greenhouse gases emitted from these trash bins and dumpsters. It is just one trash bin as it relates to pounds of emissions however multiply that by 225 Million and it becomes a significant number of emissions each day.

Furthermore, there are many other uses or embodiments of the device. The device is also highly effective for usage in personal, public or rideshare vehicles for sanitizing and deodorizing. The devices capability to exhaust dense vapor quickly affords drivers the advantage of fast and touchless sanitation while the vehicle is parked and or in between customers/riders drop offs and pick ups. The inclusion of the clock-based timer feature makes the device highly suitable for usage in cars and boats while covered or in storage. The device can be set for weekly or bi-weekly activation to eradicate and repel insects and animals that often seek haven in these stored structures causing damage. The versatility, compact and lightweight design of the device also allows for mounting almost anywhere and for the utilization of non-traditional agents such as cayenne pepper extract for repelling would be home invaders or burglars by mounting above doorways or in specific rooms in homes. The same versatility allows for connection to doorbell cams or home security camera systems for remote activation from homeowners or business owners mobile device during a break in event.

Alternatively summarized, the invention in various embodiments is a sanitizing agent vaporization and dispersal device comprising: a vaporizing unit comprising a case, a fluid tank structured to retain a liquid sanitizing agent, an air pump, a vapor diffuser comprising a venturi, a control board having a microprocessor, and batteries; wherein said vaporizing unit transitions the sanitizing agent from a liquid phase to a vapor phase, said air pump dispersing the sanitizing agent through said venturi. Furthermore, such device with said fluid tank comprising one or more wick members and a heating element; and/or said fluid tank further comprising an open-celled material to retain the sanitizing agent. Alternatively still, the invention in various embodiments is sanitizing agent vaporization and dispersal device comprising: a vaporizing unit comprising a case, one or more fluid tanks structured to retain a liquid sanitizing agent, one or more vortex generators, a control board having a microprocessor, and batteries; wherein said vaporizing unit transitions the sanitizing agent from a liquid phase to a vapor phase, said one or more vortex generators dispersing the sanitizing agent. Furthermore, such device wherein said one or more fluid tanks comprises an air inlet and a refilling port; wherein said vaporizing unit further comprises visible and audible status indicators; further including a communication technology chosen from the group of communication technologies consisting of RF, Bluetooth and Wifi; further comprising a smart device in communication with said vaporizing unit through said communication technology; wherein said case is structured with at least two sloped or angled edges; further comprising a mounting bracket to affix said vaporizing unit to an interior wall of a closed structure; and/or wherein the closed structure is a trash bin.

### BRIEF DESCRIPTION OF THE DRAWINGS

FIG. 1 is a perspective view of a first embodiment of the invention.

FIG. 2 is a plan view of the embodiment of FIG. 1.

FIG. 3 is an exposed view of FIG. 2 showing the interior elements of the embodiment.

FIG. 4 is an exploded perspective view of a second embodiment of the invention.

FIG. 5 is a rear exploded perspective view of the embodiment of FIG. 4.

FIG. 6 is a perspective view of a fluid tank of the embodiment of FIG. 4.

FIG. 7 is a view of a control board for the embodiment of FIG. 4.

FIG. 8A is a left side partial view of a schematic diagram for the control board for the embodiment of FIG. 4, to be viewed in conjunction with FIG. 8B.

FIG. 8B is a right side partial view of a schematic diagram for the control board for the embodiment of FIG. 4, to be viewed in conjunction with FIG. 8A.

FIG. 9 illustrates a representative smart device.

### DETAILED DESCRIPTION OF INVENTION

With reference to the drawings, embodiments of the invention will now be described in detail. As used herein, the term “vaporization” or related grammatical versions of the term shall be taken to mean that a sanitizing agent undergoes a phase transition, e.g., a change from the liquid phase to the vapor phase through evaporation, a surface phenomenon, or boiling, a bulk phenomenon. Thus, the invention is limited to dispersion of the agent by vaporization, as opposed to devices dispersing an agent via aerosol (use of a propellent gas), atomization (formation of droplets) or pressurization (use of compressed gas), all of which disperse liquid droplets of differing sizes, resulting in non-continuous surface coverage.

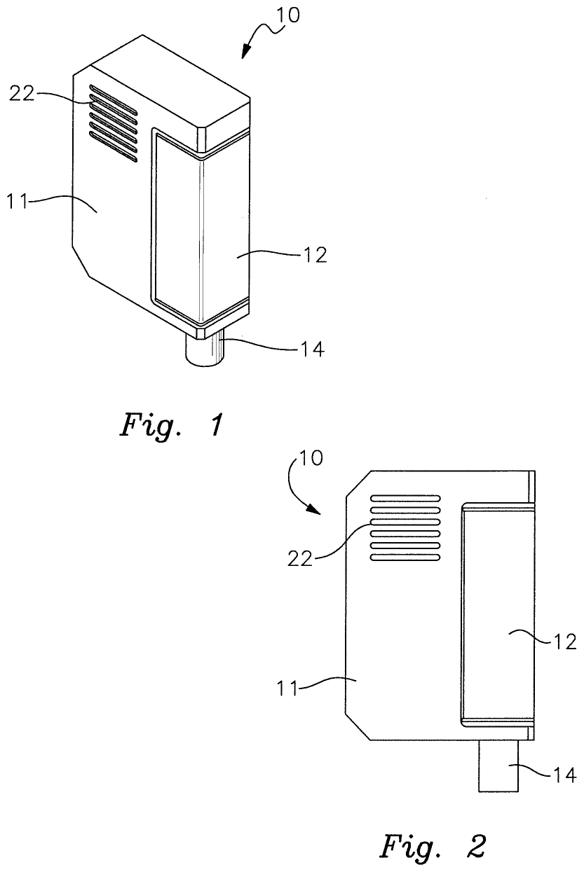

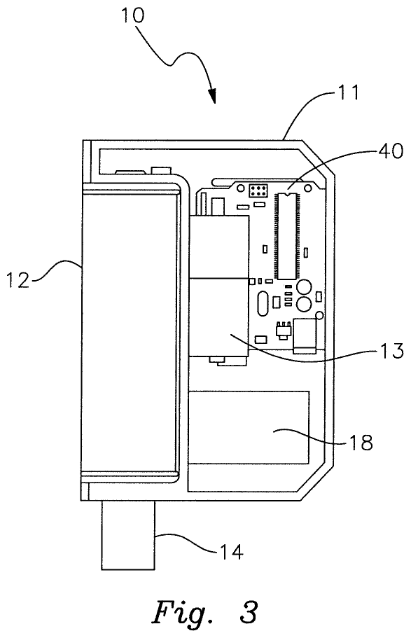

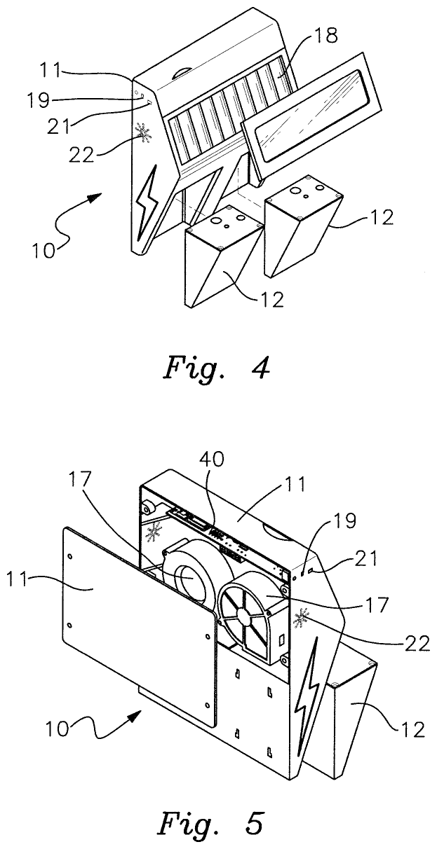

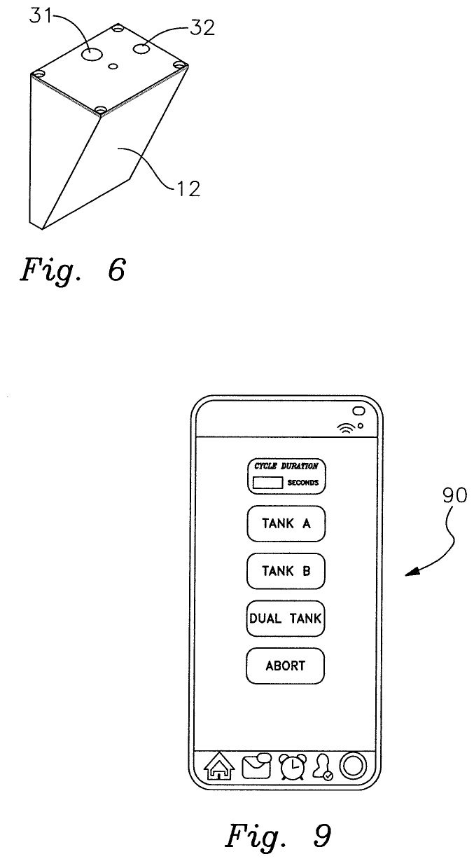

A basic embodiment of the device is shown in FIGS. 1-3 and an alternative embodiment of the device customized for use on trach bins is shown in FIGS. 4-8. The vaporizing unit 10 may be mounted to the interior of closed structures, such as rooms, vehicles, containers, receptacles, trash bins, dumpsters, shipping containers, portable decontamination boxes and any similar such structures, to be referred to herein collectively as “closed structures”. Preferably the vaporizing unit 10 is mounted to an interior surface of the closed structure, either permanently or removably, by adhesive, mechanical interlock or fasteners, and most preferably by providing a frame or mounting bracket to be affixed to the closed structure, with the vaporizing unit being then affixed to the frame or mounting unit.

The vaporizing unit 10 of FIGS. 1-3 comprises a housing, cover or case 11 which contains the operational components of the device. The case 11 is preferably configured and sized to occupy minimal space within the closed structure and to provide minimal obstruction when inserting or removing items from the closed structure. The vaporizing unit 10 comprises a removable fluid tank 12, which retains a liquid sanitizing agent, which as explained may consist of a disinfecting, sanitizing and/or barrier composition, to be referred to herein collectively as “sanitizing agents”, an air pump 13 to draw air into the case 11 through air intake ports 22 to deliver into the fluid tank 12, and a venturi port 14 for dispersing the vaporized sanitizing agent into the closed structure. The vaporizing unit 10 is controlled by a control board 40 and powered by batteries 18. Power is supplied to a heating element, such as a coil heater, retained within fluid tank 12, which heats the liquid sanitizing agent, which may be maintained within a cotton ballast or similar open-celled material, such that liquid on one or more cotton tubular members or wick members also disposed within the fluid tank 12 deliver the sanitizing agent to the heating element where it transitions to a vapor and is dispelled from the fluid tank 12 and case 11 through the venturi port 14, which acts a vapor diffuser to maximize distribution of the vaporized sanitation agent within the closed structure.

As mentioned previously, with respect to disclosing the invention in an embodiment for use wherein the closed structure is a trash bin, the case 11 is preferably configured with at least two sloped or angled edges, as shown best in the alternative embodiment of FIGS. 4-5. Furthermore, the design, geometry and dimensions of the frame or mounting bracket would be customized to enable attachment to all three major brands and sizes of curbside trash roller bins (Toter, Schaefer and Uline) as well as to any commercially available trash bin or dumpster. All these trash bins have a different design regarding the interior surface configuration requiring a mounting bracket with a very specific design to be able to attach to all of them without specialize mounting brackets for each type. The vaporizing unit 10 is inserted into the frame and the device is ready for operation. The design, geometry and dimensions of the vaporizing unit frame and cover 11 are of critical nature to vaporizing unit's 10 efficacy as well. The compact design allows for installation in any size trash container of any interior wall design or configuration. The design of the sloping sides of the cover 11 and the design of the vapor diffusors are critical to efficacy. These designs allow for the unit 10 to be affixed to the back inside upper wall of the trash bin, i.e, the door hinge side. It is crucial that the unit 10 be able to affix to this area on the trash bin for two reasons. First, the unit 10 should be affixed high enough so that agent vapor settles over and circulates around all materials in the container during fogging, and second, the unit 10 should be designed to allow bin contents to slide or fall out of the trash bin when emptied by management service trucks without damage to or catching on the unit 10. Trash bins are uniquely designed to allow autonomous or semi-autonomous dumping. All current truck systems pick up the trash bin by the front side attachment so when turned upside down to evacuate the contents the contents slide along the inside front wall of the trash bin. Additionally, the unique design of the unit's cover incorporates sloped edges on all sides. This allows contents while being dumped to slide over unit and not catch creating an issue in evacuating all the contents and or damaging the unit when the trash bin is full. Specialized seals have also been designed so that trash, debris, or fluids cannot infiltrate between the vaporizer unit and the wall of the trash bin as well as seals which render the vaporizer unit watertight.

Upon activating the vaporizing unit 10, the heating element instantly heats up and the fluid on the wick starts to vaporize. Once the liquid is vaporized an in-line vortex generator 17 creates vapor velocity and trajectory by diffusors for optimal dispersion and fog vortex. This vortex is highly important for full engulfment of all surfaces such as boxes, envelopes, trash, and trash bags surfaces. The unit 10 then automatically goes into sleep mode after the end of cycle. Temperature of the heating element is constantly monitored and kept at the desired temperature range for the entire duration of cycle. The fluid tank 12, cotton ballast, wick, volume sensors, and heating element are designed as a one-part replaceable unit. The above-mentioned functionalities possess several advantages over conventional vaporizing devices. Firstly, this allows the unit 10 to operate in any position. The tank 12/wick/heating element operational efficacy and sealing is not contingent on position or gravity. Since the logic is preferably based on feedback rather than a timer, it affords control over the circuit and eliminates the risk of over-heating or underheating of the heating element which could either burn up both wick and the heating element or cause dry hit, rendering the device useless, as is common in other vaporizer units. This in turn also ensures optimum power consumption which enhances the life of battery 18.

The device includes an on-off switch 21 and is self-sufficient as it is equipped with visual and audible status indicators 19 and notifies the user if the device needs any attention, like refill or replacement of tank units 12 or replacement of batteries 18. Furthermore, once paired with a smart device 90, the user can receive notifications directly to their smart device(s) 90 and can even configure and control the vaporizing unit 10 directly from their smart device 90. Each fluid tank unit 12 comprises an air inlet 31 and a refill port 32, as well as access means to supply power to the heating element, and is capable of being refilled multiple times. The novel approach of incorporating the fluid tank 12, with its ballast, wick, sensors, and heater element, into a compact replaceable unit 12 allows the user to easily remove the old unit 12 and snap in a new unit 12 with ease when it reaches the end of design life. This may be from six months to six years depending on usage frequency. The tank 12, ballast, wick, and heater inclusive design also significantly increases the ease of customer support as if a problem arises in the vaporizing unit's 10 operation and which requires a replacement tank 12 to be sent, and allows the user to have as a back-up a completely filled tank unit 12 to simply plug and play. Refill bottles of all sanitizing agents may be provided. This will be a refill bottle with a specially designed plunger to allow it to mate with the vaporizer unit tank refill port 32 or a no spill filling. This design also prohibits unit owners from filling tanks 12 with generic agents and or agents that could be hazardous or toxic if vaporized in the unit 10.

The intent and design of the unit 10 also provides for sale of pre-filled tank units 12 complete with the agent of choice as well as being inclusive of ballast, wick, heater, and sensors as described above. This design will significantly decrease the need for the sale of individual parts and extensive repair manuals. The design of this vaporizer unit 10 is so that it is completely compartmentalized with the benefit and safety of the user in mind.

The intent of the unit is to only vaporize ‘green’ ‘organic’ and ‘environmentally friendly’ ‘non-hazardous’ agents including but not limited to natural essential oils such as food grade Thymol, Peppermint, Cedarwood as well as low but effective concentrations of citric acid, acetic acid vinegar, or silver ions. These oils and extracts are constituted/diluted and suspended in food grade vegetable glycerin and or propylene glycol. The concentration/ratio of these agents to the suspension fluid varies for each component based on tested effectiveness. The agents are suspended in the glycol or glycerin to provide a dense vapor that will not only fully engulf all shapes and surface textures/profiles but to ‘cling’ to these surfaces. This provides both the creation of time without evaporation of agent on the surface to assure eradication of the contaminant but also allows for a barrier of the agent to remain on the surface to arrest the containments' ability to transmit. The invention is not intended to use toxic or harmful components as used in many of the other disinfectants.

The agents designed for use in this invention are proven in laboratory testing to naturally sanitize and kills 99.99% of all disease-causing bacteria, and destroy viruses created from household waste or viruses and infectious disease found and transmitted by other means. Representative sanitizing agents are Thymol and anhydrous citric acid. Both agents are currently listed on the EPA “N” list for disinfectants/pesticides approved for the eradication of the COVID 19 virus.

In addition, beyond the eradication of diseases, the sanitizing agent may be chosen to control all odors caused by household waste while naturally repelling flies, fruit flies, bees, maggots and other insects attracted to odor. Furthermore, the agent may be one chosen to naturally kills these insects and their larvae as well as providing a lasting barrier to repel them. Larger nuisance animals such as raccoons, opossums, bears etc. may also repelled by a specific agent in the vapor's lingering odor and barrier.

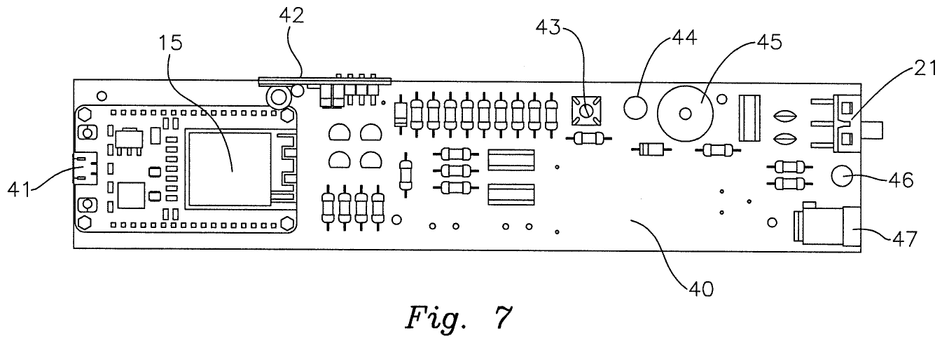

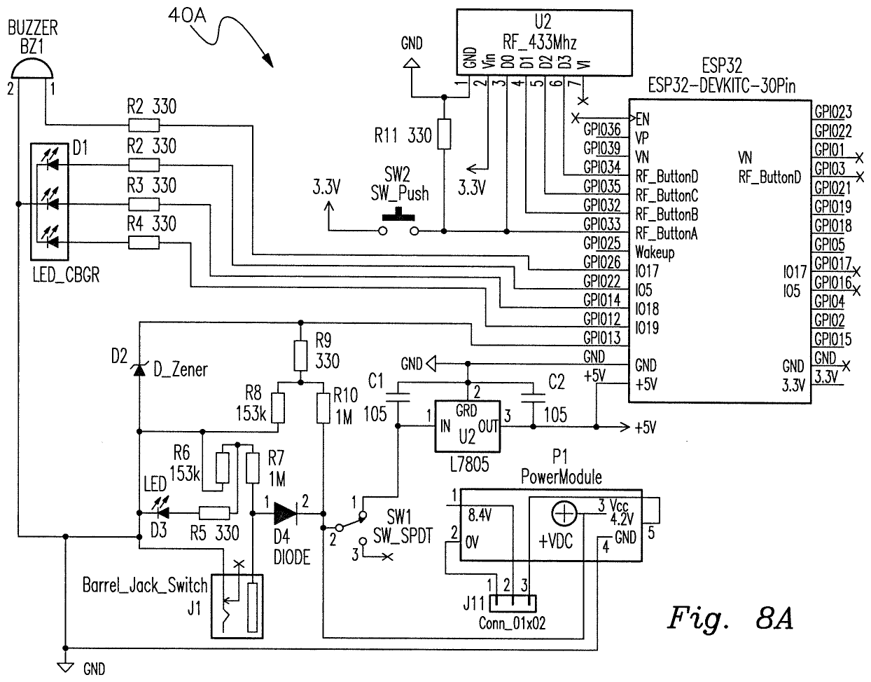

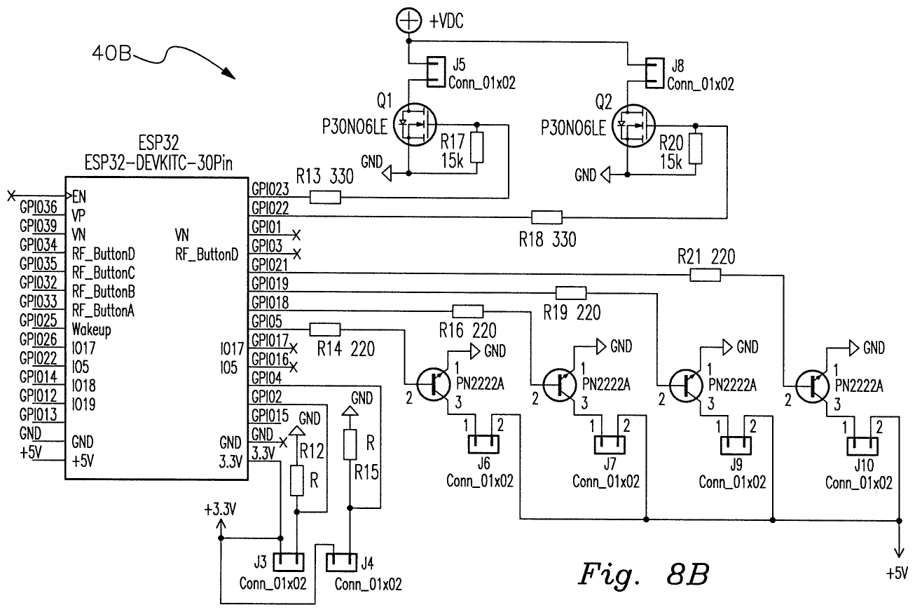

The invention incorporates novel control of component functionality, safety, and management of the electronic components with a microprocessor or micro-controller 15 mounted on the control board 40, the processor 15 running a custom program. As seen in FIGS. 7 and 8, the control board 40 may be constructed to retain in communicating manner a communication port 41, an RF module 42, a wakeup button 43, an RGB LED 44, a buzzer 45, a blue LED 46 and power jack 47.

The program and processor 15 provide multiple safety features include the use of a photocell. By taking feedback from the photocell the processor 15 can determine if the lid of the containment unit is closed. This ensures the device to work more efficiently as it prevents useless dispersion of the vapor into the ambient atmosphere. Furthermore, it also prevents the situation of vaporizing liquids and dispersing them potentially towards the user. Although the vapor is completely non-hazardous to humans and animals most all-natural agents are not recommended for long term inhalation. The device has distinguishable audible alarms 19 to let the operator/owner know prior to, during and after the fogging process is in operation. This affords just one more safety benefit to the consumer. A second safety and convenience feature are the prevention of no ‘dry firing’ of wick—hence damaging wick and or heater which is a common failure event for current vaporizer technologies. This is achieved by using a moisture sensor. In the situation of lack of moisture not only will the unit not energize the heating element, but it also energizes a low decibel alarm to alert the user. If paired with the smart device 90, all these alert notifications will be directed to user's smart device 90. In this event the user would attempt to repair the unit 10 per provided directions or simply remove the tank unit 12 and replace with a new one. The device 10 may also be equipped with an on-board charging circuit and provided with a wall charger. This add-on circuit helps in charging the batteries 18 without having the need to remove the batteries 18 and also protects the batteries 18 from overcharge/over discharge. The microcontroller 15 is integrated with the batteries 18 and constantly checks for battery level and lights different color LEDs to indicate the level of charge. The device 10 preferably automatically enters the safe mode when it detects low charge, while in this mode, the device 10 does not accept any command until the batteries 18 are charged to a minimum of mid-level. Moreover, upon detecting low charge, the device 10 continuously flashes red light for up to 30 seconds and enters the deep sleep mode to save power. Once fully charged, the device 10 is ready to accept any command sent using the remote or smart device 90. The battery level can also be monitored using a Bluetooth app and the user can get notifications directly on their smart devices 90 about the battery health.

The device 10 has multiple modes of operation for the user's convenience. The first method of triggering the vaporizer unit 10 is via an integrated remote-control and RF sensor for manual operation only. The unit 10 preferably includes an integrated real time clock allowing the user to set a schedule and duration of activation. This timer can also be set based on the size of the closed structure being used assuring the closed structure is fully engulfed and assuring that the unit 10 does not operate longer than is required to eradicate the virus or other intended target. The user can set the device 10 to vaporize at specific times and intervals. Other modes of control utilize an integrated Bluetooth and Wi-Fi chip. To control via Bluetooth the user installs an application on their smart device 90. Once within range of the unit 10 the user can manually activate the device 10, set the activation schedule, and ensure there are no fluid level alarms. If a Wi-Fi network is available the device 10 can be connected to internet. With this connectivity the user can utilize a custom application on their internet connected mobile device 90 to remotely monitor and control the device 10. The device 10 is also preferred to be accessible through any internet connected device via web browser. With Wi-Fi the user can also set up email alerts so that fluid level alerts are sent directly to their email of choice.

It is understood that equivalents and substitutions for certain elements set forth above may be obvious to those of ordinary skill in the art, and therefore the true scope and definition of the invention is to be as set forth in the following claims. The embodiments and illustrations are meant to be of a representative, non-limiting nature, as are the descriptions set forth above.

I claim:

1. A sanitizing agent vaporization and dispersal device comprising:
a vaporizing unit comprising a case, a fluid tank structured to retain a liquid sanitizing agent, an air pump, a vapor diffuser comprising a venturi, a control board having a microprocessor, and batteries;
wherein said vaporizing unit transitions the sanitizing agent from a liquid phase to a vapor phase, said air pump dispersing the sanitizing agent through said venturi.

2. The device of claim 1, said fluid tank comprising one or more wick members and a heating element.

3. The device of claim 2, said fluid tank further comprising an open-celled material to retain the sanitizing agent.

4. A sanitizing agent vaporization and dispersal device comprising:
a vaporizing unit comprising a case, one or more fluid tanks structured to retain a liquid sanitizing agent, one or more vortex generators, a control board having a microprocessor, and batteries;
wherein said vaporizing unit transitions the sanitizing agent from a liquid phase to a vapor phase, said one or more vortex generators dispersing the sanitizing agent.

5. The device of claim 4, wherein said one or more fluid tanks comprises an air inlet and a refilling port.

6. The device of claim 4, wherein said vaporizing unit further comprises visible and audible status indicators.

7. The device of claim 4 further including a communication technology chosen from the group of communication technologies consisting of RF, Bluetooth and Wifi.

8. The device of claim 7, further comprising a smart device in communication with said vaporizing unit through said communication technology.

9. The device of claim 4, wherein said case is structured with at least two sloped or angled edges.

10. The device of claim 9, further comprising a mounting bracket to affix said vaporizing unit to an interior wall of a closed structure.

11. The device of claim 10, wherein the closed structure is a trash bin.
